(19) United States
(12) Patent Application Publication
(10) Pub. No.: US 2022/0105321 A1
(43) Pub. Date: Apr. 7, 2022

(54) PRESSURE REGULATOR FOR VERIFYING THE INFLATION OF BALLOONS OF ENDOTRACHEAL CANNULAS OR TUBES

(71) Applicant: Héctor De Jesús Velez Rivera, Zapopan, Jalisco (MX)

(72) Inventor: Héctor De Jesús Velez Rivera, Zapopan, Jalisco (MX)

(21) Appl. No.: 17/040,847

(22) PCT Filed: Mar. 26, 2018
(86) PCT No.: PCT/IB2018/052059

§ 371 Date: Feb. 10, 2021

(51) Int. Cl. A61M 25/10; G05D 16/10; A61M 16/04; A61M 16/20; A61M 16/00

(52) U.S. Cl. CPC A61M 25/10187; G05D 16/103; A61M 2016/0027; A61M 16/208; A61M 16/0003; A61M 16/0402

(57) ABSTRACT

The invention describes a pressure regulator for verifying the inflation of balloons of endotracheal cannulas or tubes, the regulator comprising: a housing with an inlet and an outlet in which an endotracheal cannula or tube connector that includes the balloon is coupled. The housing has a flow chamber and a pressure regulating chamber; a one-way valve mounted on the inlet; the housing also comprises a plurality of deflectors mounted in the flow chamber to reduce the velocity of the gaseous fluid. The regulator has a pressure-indicating piston mounted in a movable manner in the pressure-regulating chamber, which is visible to the user; a sealing piston operationally connected with the indicating piston and underneath same so that they both move simultaneously.

### FIELD OF THE INVENTION

The present invention refers to technics used in the design and construction of apparatuses and devices for medical use and, more particularly, it is related to a pressure regulator for balloons of endotracheal cannulas or tubes which automatically avoids having a pressure greater than a preset pressure in the balloon for the proper intubation of a patient.

### BACKGROUND OF THE INVENTION

The endotracheal cannulas or tubes are commonly used for handling the respiratory tracts, particularly, during general anesthesia, critical cares, mechanical ventilation and/or urgency medicine. Among the main components of the endotracheal cannulas or tubes there is a connector being an intermediate piece between the cannula or tube and a respirator or gases source directed to the patient; the connector normally is a standard mechanic piece having a Luer-type connection well known for one skilled in the art.

The main portion of the cannulas or tubes is the body itself, a conduit or an enlarged tube carrying the gas flow from the source providing the gases to the patient. The tube normally has a circular cross section.

Now then, near the distal end of the body and externally wrapping the same an inflating portion known as a balloon, cylinder or pneumo is provided, which when inflated exerts pressure on the tracheal mucosa for the proper positioning of the cannula or tube inside the patient. In this regard, the pressure inside the balloon must be kept in certain values, as for example 25 cm of H₂O. For inflating and regulating the pressure of the balloon, manual actuation valves are used, in this concern, it is worth noting to mention the pressure in the balloon must be kept in optimal values for avoiding damage to the patient.

From the above, it is clearly appreciated that, during intubation, the medical staff performs two tasks, one of such tasks is carefully placing the cannula or tube inside the patient and the other is inflating and regulating the pressure in the balloon which may consume time and distract the personal staff from other important tasks as the anesthetic procedure.

### BRIEF DESCRIPTION OF THE INVENTION

In order to overcome the problems of the state of the art, a pressure regulator for verifying the inflation of the balloons of endotracheal cannulas or tubes has been developed, the regulator comprising the following main elements: a housing including an inlet for feeding a gaseous fluid and an outlet for coupling a cannula connector or endotracheal tube including a balloon around a portion of its distal end. The housing is divided into a flow chamber and a pressure regulating chamber.

The present invention provides a one-way valve mounted in the inlet for only allowing the entrance of gaseous fluid to the flow chamber and avoiding leaks or return of the gaseous fluid, thus reducing the pressure required in the balloon.

In the pressure regulator, a plurality of deflectors mounted in the flow chamber for reducing the velocity of the gaseous fluid within said chamber is provided, in this sense, the plurality of deflectors defines a flow pathway between the inlet and the outlet, wherein the outlet is in flow communication with both the flow chamber and the pressure regulating chamber.

In a preferred embodiment, the plurality of deflectors comprises vertical deflectors and horizontal deflectors such that a horizontal deflector is followed by a vertical deflector. Even more preferred, the deflectors positioned in a horizontal manner partially cover the cross section of the flow chamber. On the other hand, the flow chamber has a front wall and a rear wall, such that each of the horizontal deflectors is internally separated from both the front wall as well as the rear wall such that the gaseous fluid flows due the separation between the deflector and said front wall or rear wall.

In addition, in the present invention, it is important that the medical staff recognizes and notes whether a preset pressure value has been exceeded, and therefore it is provided a pressure indicating piston movably mounted in the pressure regulating chamber and visible by the user, also, there is a sealing piston operatively connected to the indicating piston and underneath the same, such that both may be simultaneously moved.

In the housing, a wall for separating the flow chamber from the pressure regulating chamber is provided. The pressure regulating chamber has an upper wall, preferably transparent, in which it is visible the indicating piston in case the same is upwardly moved by exceeding the preset pressure value in the balloon, from other point of view, the transparent wall allows to view whether the balloon is inflated at the proper pressure.

In order to relieve the pressure in case of an overpressure in the balloon, at least one relief orifice localized in the pressure regulating chamber is provided, said orifice communicates the interior of the housing with the ambient exterior, and said orifice is blocked by the sealing piston when the pressure in the balloon is less than the predetermined value. Also, it is preferred that the regulating chamber has a front wall and a rear wall, including each wall at least one relief orifice. More particularly, it is preferred to provide a set of at least three orifices both in the front wall and other set in the rear wall, wherein the orifices of each set are horizontally arranged and equidistant one to respect the other.

The present invention also includes tension media comprising a base or anchor mounted to the housing and a flexible element connecting the base with the sealing piston such that the flexible element is tensed for maintaining the sealing piston in a blocking position when the pressure in the balloon is less than such preset value and in case there is a higher pressure in the balloon, the tension in the flexible element is overcome for upwardly moving the sealing piston and in such way upwardly moving the indicating piston in order to release said at least one relief orifice whereby the gaseous fluid may escape thereby returning to the pressure required in the balloon.

### BRIEF DESCRIPTION OF THE FIGURES

The novel aspects which are characterizing of present invention will be now established, particularly to the attached claims. However, the invention together with other objects and advantages thereof should be better understood in the underneath detailed description of some preferred embodiments of the invention, when reading together with the figures, in which:

FIG. 1 is a front view of the pressure regulator for verifying the inflation of the balloons of the endotracheal cannulas or tubes constructed according to a preferred embodiment of present invention.

FIG. 2 is a bottom view of the pressure regulator of FIG. 1.

FIG. 3 is a first cutaway view along line A-A′ of FIG. 1.

FIG. 4 is a perspective view along line A-A′ of FIG. 1.

### BRIEF DESCRIPTION OF THE PREFERRED EMBODIMENTS OF THE INVENTION

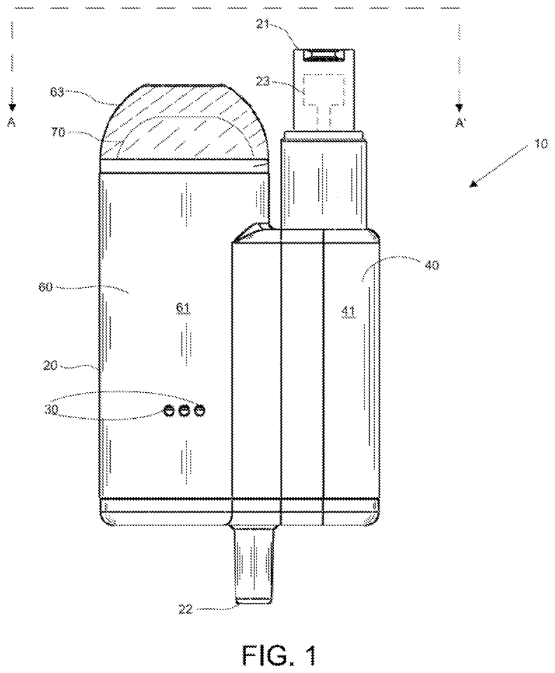

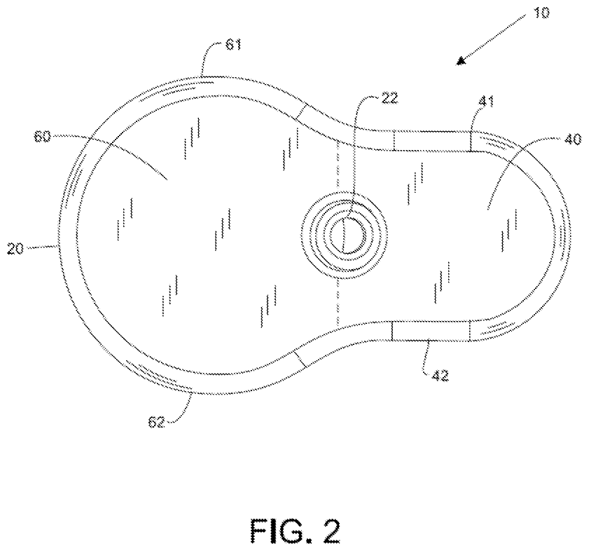

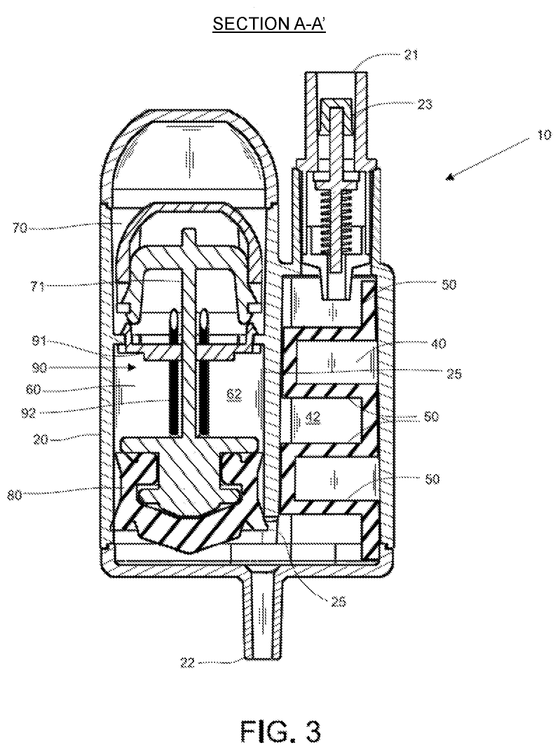

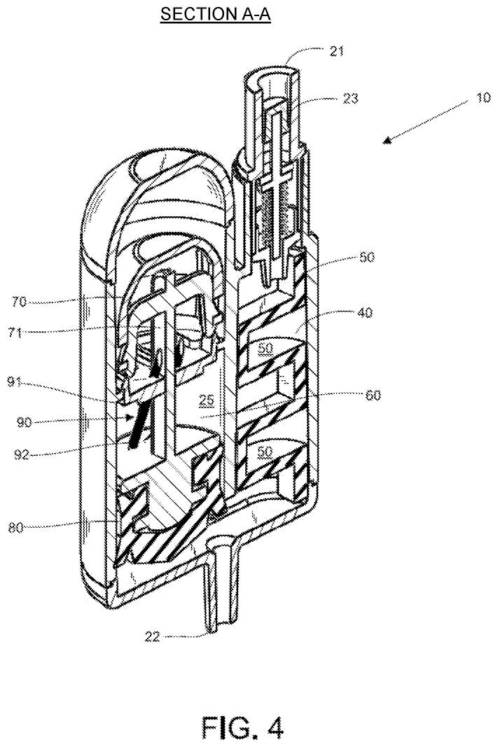

Referring to FIGS. 1 to 4 of the attached drawings, they show, a pressure regulator 10 for verifying the inflation of the balloons of the endotracheal cannulas or tubes, in present invention the term cannula or tube refers to the same object and therefore this terminology is indistinct in present invention, the pressure regulator 10 comprising: a housing 20 including an inlet 21 for feeding a gaseous fluid and an outlet 22 in which a cannula connector or endotracheal tube including the balloon (not shown) is coupled. The housing 20 is divided in a flow chamber 40 and a pressure regulating chamber 60. The regulator 10 includes a one-way valve 23 mounted in the inlet 21 to only allow the entrance of the gaseous fluid to the flow chamber 40 and avoiding leaks or the return of the gaseous fluid, thus reducing the pressure of the balloon.

Inside the housing 20, it is included a separation wall 25 to divide the housing in two portions separating the flow chamber 40 from the pressure regulating chamber 60. The outlet 22 is in flow communication with both the flow chamber 40 and the pressure regulating chamber 60.

Inside the housing 20 there is also a plurality of deflectors 50 mounted in the flow chamber 40 for reducing the velocity of the gaseous fluid within the flow chamber 40, the plurality of deflectors 50 define a flow pathway between the inlet 21 and the outlet 22.

Inside the pressure regulating chamber 60, there is an indicating piston 70 of pressure movably mounted in the pressure regulating chamber 60 and which is visible by the user; inside said regulating chamber 60 there is also a sealing piston 80 operatively connected to the indicating piston 70 and underneath the same such that both are simultaneously moved. To that end, pistons 70 and 80 are connected therebetween via a post 71.

The pressure regulating chamber 60 has an upper wall 63 being transparent to view the indicating piston 70 in case the same is upwardly moved, such that the indicating piston and the upper wall allow knowing whether the balloon has the required pressure. Said upper wall 63 having a curved shape wall with a flatten upper end, but the same may have any other shape in order to be visible by the user.

Now then, the device 10 also comprises at least one pressure relief orifice 30 provided in the pressure regulating chamber, said orifice 30 being blocked by means of the sealing piston when the pressure in the balloon is less than a predetermined value.

An important feature of present invention is the fact that the same comprises tension media 90 comprising a base 91 mounted to the housing 20 and a flexible element 92 connecting the base 91 to the sealing piston 80 such that said flexible element 91 is initially tensed for maintaining the sealing piston 80 in a blocking position when the pressure in the balloon of the endotracheal cannula is less than a predetermined pressure value and in case there is a higher pressure in the balloon, the tension in the flexible element 92 is overcome to allow an upward movement of the sealing piston 80 and consequently upwardly moving the indicating piston 70 to release said relief orifice 30.

Describing in greater detail the flow chamber 40, the deflectors 50 comprise vertical and horizontal deflectors such that a horizontal deflector is followed by a vertical deflector; from them, the deflectors positioned in a horizontal manner partially cover the cross section of the flow chamber 40.

The flow chamber 40 has a front wall 41 and a rear wall 42, such that each of the horizontal deflectors is internally separated from the front wall 41 and the rear wall 42 such that the gaseous fluid softly flows by the separation between the deflector and said front or rear wall.

On the other hand, the pressure regulating chamber 60 also has a front wall 61 and a rear wall 62, including each of these walls at least one relief orifice 30. More specifically said relief orifices 30 are provided as a set of three orifices 30 in both the front wall 61 and the rear wall 62 of the pressure regulating chamber. The orifices 30 of each set are horizontally arranged and equidistant each to respect the other.

According to the previously disclosed, it is evident for one skilled in the art that the embodiment of the pressure regulator above disclosed is just for illustrative purposes since one skilled in the art may perform numerous variations thereof, when and if they are designed with the principles of present invention. Consequently, from the above, the present invention includes all the embodiments that one skilled in the art may pose from the concepts contained in present description, according with the following claims.

### REFERENCE LIST

10 Pressure Regulator
20 Housing
21 Inlet
22 Outlet
23 Valve
25 Separation Wall
30 Relief Orifices
40 Flow Chamber
41 Front Wall
42 Rear wall
50 Deflectors
60 Pressure Regulating Chamber
61 Front Wall
62 Rear wall
63 Upper Wall
70 Indicating piston
71 Post
80 Sealing piston
90 Tension Media
91 Base
92 Elastic Element

What is claimed is:

1. A pressure regulator for verifying the inflation of balloons of endotracheal cannulas or tubes, comprising:
a housing including an inlet for feeding a gaseous fluid and an outlet wherein a cannula connector or endotracheal tube including the balloon is coupled; the housing being divided in a flow chamber and a pressure regulating chamber;
a one-way valve mounted in the inlet for only allowing the entrance of the gaseous fluid to the flow chamber and avoiding leaks or the return of the gaseous fluid that decrease the pressure in the balloon;
a plurality of deflectors mounted in the flow chamber for reducing the velocity of the gaseous fluid inside the flow chamber, defining the plurality of deflectors a flow pathway between the inlet and the outlet;
a pressure indicating piston movably mounted in the pressure regulating chamber and which is visible by the user;
a sealing piston operatively connected to the indicating piston and underneath the same such that both are simultaneously moved;
at least one pressure relief orifice provided in the pressure regulating chamber, being said orifice blocked by the sealing piston when the pressure in the balloon is less than a predetermined value;
tension media comprising a base mounted to the housing and a flexible element connecting the base with the sealing piston such that said media maintain the sealing piston In a blocking position when the pressure in the balloon of the endotracheal cannula or tube is less than said predetermined value and in case there is a higher pressure in the balloon, the tension in the flexible element is overcome for upwardly moving the sealing piston and consequently the indicating piston is upwardly moved to release said at least one pressure relief orifice.

2. The pressure regulator according to claim 1, wherein the plurality of deflectors comprises vertical deflectors and horizontal deflectors such that a horizontal deflector is followed by a vertical deflector.

3. The pressure regulator according to claim 2, wherein the deflectors horizontally positioned partially cover the cross section of the flow chamber.

4. The pressure regulator according to claim 3, wherein the flow chamber has a front wall and a rear wall, such that each of the horizontal deflectors are internally separated from the front wall and the rear wall so that the gaseous fluid flows by the separation between the deflector and said front wall or rear wall.

5. The pressure regulator according to claim 1, wherein the regulating chamber has a front wall and a rear wall, including each of said walls at least a relief orifice.

6. The pressure regulator according to claim 5, wherein a set of three orifices is provided in both the front wall and the rear wall, and wherein the orifices of each set are horizontally arranged and equidistant each to respect the other.

7. The pressure regulator according to claim 1, wherein the housing includes a separation wall for separating the flow chamber from the pressure regulating chamber.

8. The pressure regulator according to claim 1, further comprising a post for connecting the indicating piston with the sealing piston.

9. The pressure regulator according to claim 1, wherein the outlet is in flow communication with both the flow chamber and the pressure regulating chamber.

10. The pressure regulator according to claim 1, wherein the pressure regulating chamber has a transparent upper wall such that the indicating piston is visible in case the same is upwardly moved.

11. The pressure regulator according to claim 10, wherein the upper wall has a curved shape with a flatten upper end visible by the user.
